(19) United States
(12) Patent Application Publication
(10) Pub. No.: US 2025/0143921 A1
(43) Pub. Date: May 8, 2025

(54) CIRCULATION THERAPY DEVICE WITH ADAPTIVE POWER SUPPLY-BASED CONTROL

(71) Applicant: AVENT, INC., Alpharetta, GA (US)

(72) Inventors: Bryan D. HUFF, Alpharetta, GA (US); Himanshu PATEL, Alpharetta, GA (US)

(21) Appl. No.: 18/704,751

(22) PCT Filed: Oct. 25, 2022
(86) PCT No.: PCT/US2022/047699

§ 371 Date: Apr. 25, 2024

Related U.S. Application Data
(60) Provisional application No. 63/271,510, filed on Oct. 25, 2021.

(51) Int. Cl. A61F 7/02

(52) U.S. Cl. CPC A61F 7/02; A61F 2007/0295

(57) ABSTRACT

An exemplary system and method is disclosed to configure a controller of a fluid circulation therapy device based on its connected power supply. The controller is configured to function in multiple modes with varying input power available. The power supply includes circuitries to direct the controller to select one of those modes, which can adapt the fluid circulation therapy device for one of many different environments, thermal power, and noise operations. The configuration of the fluid circulation therapy device, without additional human interaction, facilitates the efficient manufacturability of different classes of the fluid circulation therapy devices.

### RELATED APPLICATIONS

The present application claims priority to U.S. Provisional Application No. 63/271,510, filed Oct. 25, 2021, which is incorporated herein by reference for all purposes.

### BACKGROUND

Heat therapy or cool therapy are used to treat various sport injuries, post-operation, and ailments. Sophisticated heating and cooling fluid circulation therapy systems are commercially available that can provide heating or cooling therapy for different types of sports and injuries and various areas of the body. These heating and cooling therapy systems can include a compress or wrap that can apply heating and cooling therapy to localized areas of the body. The compress or wrap are connected to a heat exchanger system that provides the cooling or heating fluids. In some embodiment, the wrap can be actuated to additionally provide compression therapy to provide optimal therapeutic benefits after injury or surgery.

Indeed, there can be a large number of wrap designs for the different types of sports and injuries and various areas of the body, which can require different cooling and heating requirements. Manufacturers can design customized heat exchanger systems for a subset of these wraps and therapeutics applications.

### SUMMARY

An exemplary system and method are disclosed to configure a controller of a fluid circulation therapy device based on its connected power supply. The controller is configured to function in multiple modes with varying input power available. The power supply includes circuitries to direct the controller to select one of those modes, which can adapt the fluid circulation therapy device for one of many different environments, thermal power, and noise operations. The configuration of the fluid circulation therapy device, without additional human interaction, facilitates the efficient manufacturability of different classes of fluid circulation therapy devices.

In an aspect, a fluid circulation system is disclosed comprising a compressor configured to connect in a fluid loop with a fluid circulation cooling therapy device, including a first fluid circulation therapy device (e.g., having a first cooling capacity) and a second fluid circulation therapy device (e.g., having a second cooling capacity); a controller that is operatively coupled to the compressor, wherein the controller includes a control output coupled to a driver that is configured to energize the compressor from a power source; a detection circuit configured to determine coupling to one of a set of available power supplies, including a first power supply and a second power supply, wherein the first power supply has a first power output, and the second power supply has a second power output, wherein the first voltage output is higher than the second voltage output (e.g., having higher voltage or current to provide higher power); and the controller having instructions for a control loop of the compressor to determine the control output, wherein the control loop includes instructions to i) set a control parameter associated with a maximum compressor speed setpoint (e.g., current limit) with a first setpoint value upon detecting coupling of the first power supply and ii) set the control parameter with a second setpoint upon detecting coupling of the second power supply, and wherein the first setpoint is higher than the second setpoint.

In some embodiments, the detection circuit is configured to determine coupling to the set of available power supplies via an analog or digital output or a sensed electrical characteristic provided by the connected power supply at a power supply cable that connects the connected power supply to the detection circuit.

In some embodiments, the system's power supply cable includes a connector, the connector having wires for a first wiring configuration associated with the first power supply and a second wiring configuration associated with the second power supply.

In some embodiments, the system's power supply cable includes a connector, the connector having wires for a first wiring configuration associated with the first power supply and a second wiring configuration associated with one of a set of available second power supplies or a battery.

In some embodiments, the system includes a variable condenser fan speed, wherein the controller is coupled to the variable condenser fan speed, wherein the controller includes a second control output coupled to the driver that is configured to energize the variable condenser fan speed from the power source; and wherein the controller has instructions for a control loop of the variable condenser fan speed to determine the second control output, wherein the second control loop includes instructions to set a control parameter associated with a maximum variable condenser fan speed setpoint with a third setpoint value upon detecting coupling of the first power supply and set the control parameter with a fourth setpoint upon detecting coupling of the second power supply, and wherein the third setpoint is higher than the fourth setpoint.

In some embodiments, the control loop includes instructions to set the control parameter associated with a maximum compressor speed setpoint (e.g., current limit) with a third setpoint for operation of the compressor that minimize acoustic noise.

In some embodiments, the control loop includes instructions to limit a speed control voltage change from one cycle to the next (e.g., to prevent excessive cycling of the control voltage).

In some embodiments, the fluid loop may be cooled before circulating back into the wrap. Alternatively, the fluid may bypass the cooling source before recirculating into the heat transfer device.

In some embodiments, the cooling capacity includes cooling fluid such as tap water. Other cooling capacities such as thermoelectric, chemical, or electromechanical cooling capacities may be used as would be understood by one of skill in the art.

In some embodiments, the control output operatively coupled to the compressor may include data transmission methods including pulse width modulation (PWM), analog-digital conversion (ADC), isolation transformers, filters, and more.

In some embodiments, the control loop includes instructions to provide a delay from a time the compressor is turned off (e.g., DAC output of zero) to when it can be restarted, and instructions to provide a delay from a time the compressor is turned on to when it can be turned off.

In some embodiments, the fluid circulation cooling therapy device is configured with a first cooling capacity and is configured to be attachable to a first area of a body, and wherein the second fluid circulation cooling therapy device is configured with a second cooling capacity and is configured to be attachable to a second area of the body.

In another aspect, a system is disclosed comprising a controller configured to operatively couple to a compressor, wherein the compressor is configured to connect in a fluid loop with a fluid circulation cooling therapy device, including a first fluid circulation therapy device and a second fluid circulation cooling therapy device, and wherein the controller includes a control output coupled to a driver that is configured to energize the compressor from a power source; and a detection circuit configured to determine coupling to one of a set of available power supplies, including a first power supply and a second power supply, wherein the first power supply has a first power output, and the second power supply has a second power output, wherein the first voltage output is higher than the second voltage output (e.g., having higher voltage or current to provide higher power); and the controller having instructions for a control loop of the compressor to determine the control output, wherein the control loop includes instructions to set a control parameter associated with a maximum compressor speed setpoint (e.g., current limit) with a first setpoint value upon detecting coupling of the first power supply and set the control parameter with a second setpoint upon detecting coupling of the second power supply, and wherein the first setpoint is higher than the second setpoint.

In some embodiments, the system's detection circuit is configured to determine coupling to the set of available power supplies via an analog or digital output or a sensed electrical characteristic provided by the connected power supply at a power supply cable that connects the connected power supply to the detection circuit.

In some embodiments, the system's power supply cable includes a connector, the connector having wires for at least one of: i) a first wiring configuration associated with the first power supply and ii) a second wiring configuration associated with the second power supply; a digital communication bus to communicate with the first power supply and the second power supply; or iii) a first wiring configuration associated with the first power supply and iv) a second wiring configuration associated with one of a set of available second power supplies or a battery.

In some embodiments, the system's control loop includes instructions to set the control parameter associated with a maximum compressor speed setpoint (e.g., current limit) with a third setpoint for operation of the compressor that minimizes acoustic noise.

In some embodiments, the invention is directed to a method of providing thermal treatment including detecting, via a circuit, coupling of a power supply cable between a controller and one of a set of available power supplies, including a first power supply and a second power supply, wherein the first power supply has a first power output, and the second power supply has a second power output, wherein the first voltage output is higher than the second voltage output; setting, by a processor of the controller, a control parameter associated with a maximum compressor speed setpoint with a first setpoint value upon detecting coupling of the first power supply and ii) set the control parameter with a second setpoint upon detecting coupling of the second power supply, and wherein the first setpoint is higher than the second setpoint; and outputting, by the controller, a control output for a compressor of a fluid circulation cooling therapy system, wherein the control output is limited by the control parameter associated with a maximum compressor speed setpoint.

The system, device, and method of the present invention have other features and advantages which will be apparent from or are set forth in more detail in the accompanying drawings, which will be apparent from or are set forth in more detail in the accompanying drawings, which are incorporated in and from a part of this specification, and the following Detailed Description of the Invention, which together serves to explain the principles of the present invention.

### BRIEF DESCRIPTION OF THE DRAWINGS

The patent or application file contains at least one drawing executed in color. This application is directed to the evaluation of the field of view of a person. Evaluative scenes and results, as presented in color, may be necessary for the understanding of the claims. Copies of this patent or patent application publication with color drawing(s) will be provided by the Office upon request and payment of the necessary fee.

Embodiments of the present invention may be better understood from the following detailed description when read in conjunction with the accompanying drawings. Such embodiments, which are for illustrative purposes only, depict novel and non-obvious aspects of the invention. The drawings include the following figures.

FIG. 1 shows a diagram of a fluid circulation therapy device comprising a controller configured with adaptive controls in accordance with an illustrative embodiment.

FIG. 2A shows an example embodiments of a fluid circulation system in accordance with an illustrative embodiment.

FIGS. 2B and 2C shows example screens displayed by the touch screen interface of the fluid circulation therapy device and parameters for control logic and operation of the controller in accordance with an illustrative embodiment.

FIGS. 3A and 3B show example fluid wraps and compresses that can be used with the fluid circulation therapy device of FIG. 1 in accordance with an illustrative embodiment.

FIG. 4 shows a method to operate a circulation therapy device with adaptive power supply-based control in accordance with an illustrative embodiment.

FIGS. 5A, 5B, 5C each shows example embodiments of the connector of the controller to facilitate adaptive power supply-based control in accordance with an illustrative embodiment.

FIGS. 6A and 6B, collectively, show an example control loop and logic for the controller of FIG. 1 configured with adaptive power supply-based control in accordance with an illustrative embodiment.

FIGS. 7A, 7B, and 7C, collectively, show another example control loop and logic for the controller of FIG. 1 configured with adaptive power supply-based control in accordance with an illustrative embodiment.

### DETAILED SPECIFICATION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the reference list. For example, Ref. [1] refers to the 1^{st} reference in the list. All references cited and discussed in this specification are incorporated herein by reference in their entireties and to the same extent as if each reference was individually incorporated by reference. Example Fluid Circulation Therapy System

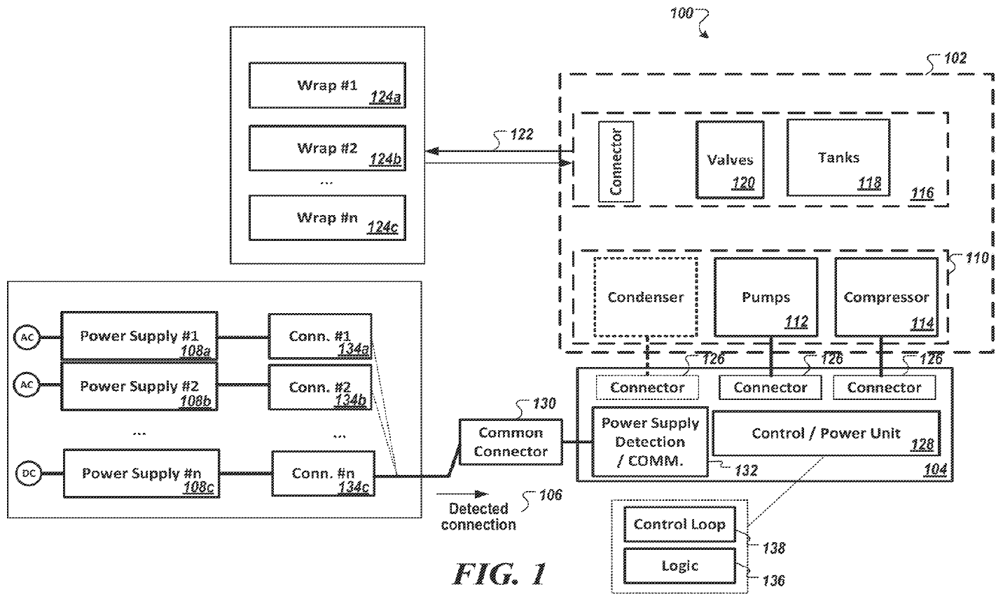

FIG. 1 shows a diagram 100 of a fluid circulation therapy device 102 comprising a controller 104 configured with adaptive controls in accordance with an illustrative embodiment. The controller 104 is configured to adaptively adjust its operating configuration (e.g., control logic, parameters, or settings) based on a detected connection 106 to a power supply 108 (shown as 108a, 108b, 108c) for the fluid circulation therapy device 102. The adaptive configuration of the controls of the fluid circulation therapy device 102 facilitates the use of a modular design and manufacturing that can be adaptively programmed with minimal manual configuration of the system software. The controller and adaptive control configuration allow multiple product configurations in a product line with few part counts that beneficially strengthen supply lines.

The fluid circulation therapy device 102 includes a base thermal regulation system 110 that is configured to include similar or the same components between different product lines. The base system 110 is coupled to one or many different available circulation systems having different configurations of tanks and valves for a different product in the product line of fluid circulation therapy devices 102. Indeed, the base system and accompanying electronics and controls can facilitate the customizability of the fluid circulation therapy device 102 with minimal component configurations.

In the example of FIG. 1, the base thermal regulation system 110 includes a pump 112 and compressor 114. The base thermal regulation system 110 operates with the circulation system 116 that includes one or more tanks 118 for water or fluid storage and valves 120. The base thermal regulation system 110 operates with the circulation system 116, in a cooling or heating loop 122, that couples to a therapeutic wrap or compress 124 (shown as 124a, 124b, 124c) to provide the therapy cooling or heating. The therapeutic wrap or compress 124 can be tailored (e.g., in terms of size, attachments, cooling or heating capacity) for specific placement on a user. In some embodiments, the cooling or heating loop 122 can provide electrical power to the wrap or compress 124 to activate compression action at the wrap or compress 124. The base thermal regulation system 110 is coupled to the controller 104 through connectors 126. Controller 104 includes a control system 128 (shown as “control/power unit” 128) that is configured to send command signals or power to the compressor device 114 and pump 112 to control the circulation system 116. The thermal regulation system 110 may include other components such as a condenser or blower as well as appropriate control and flow valves and pressure/temperature sensors.

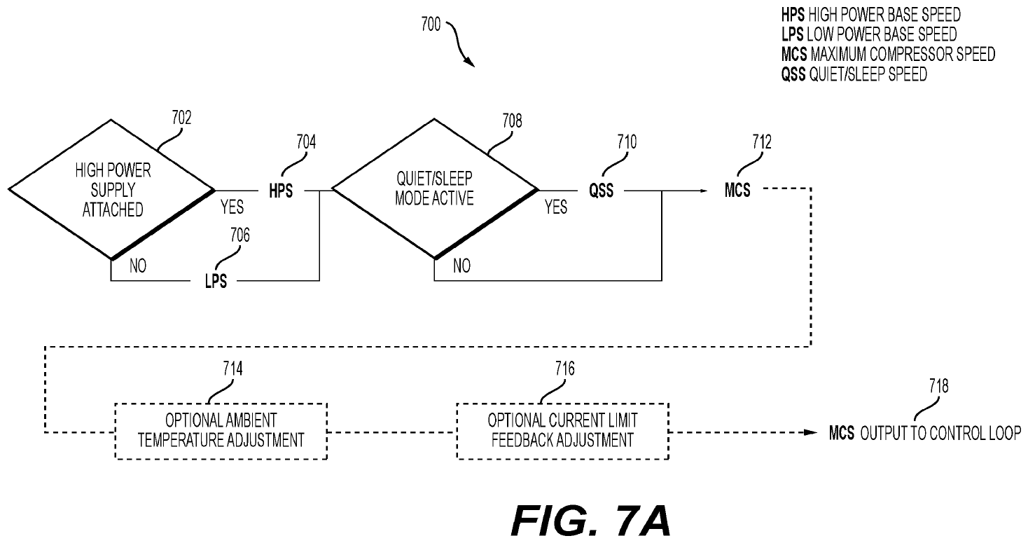

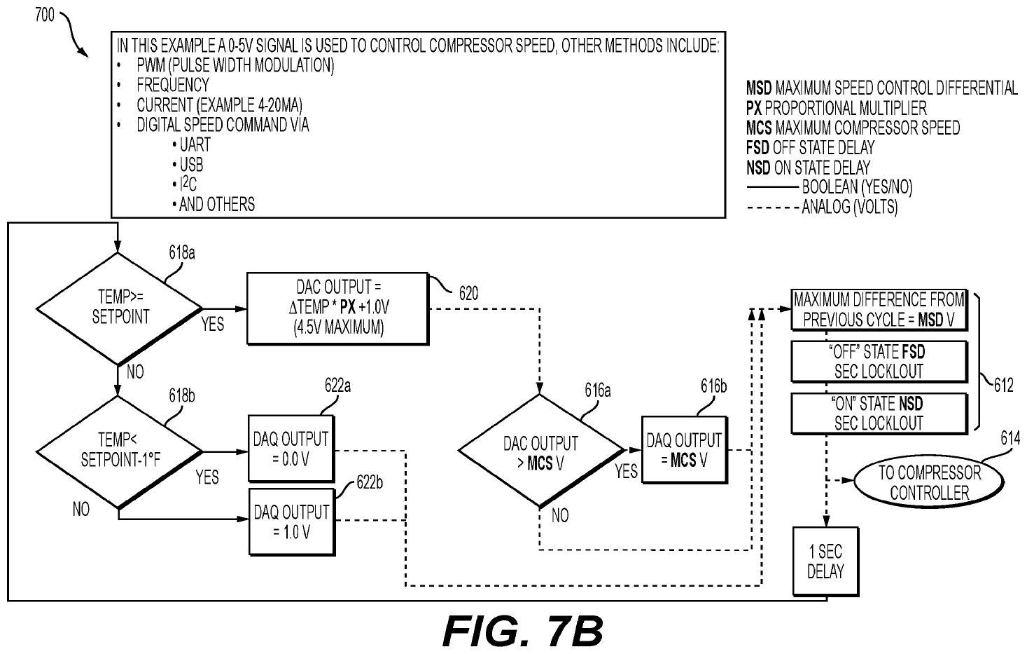

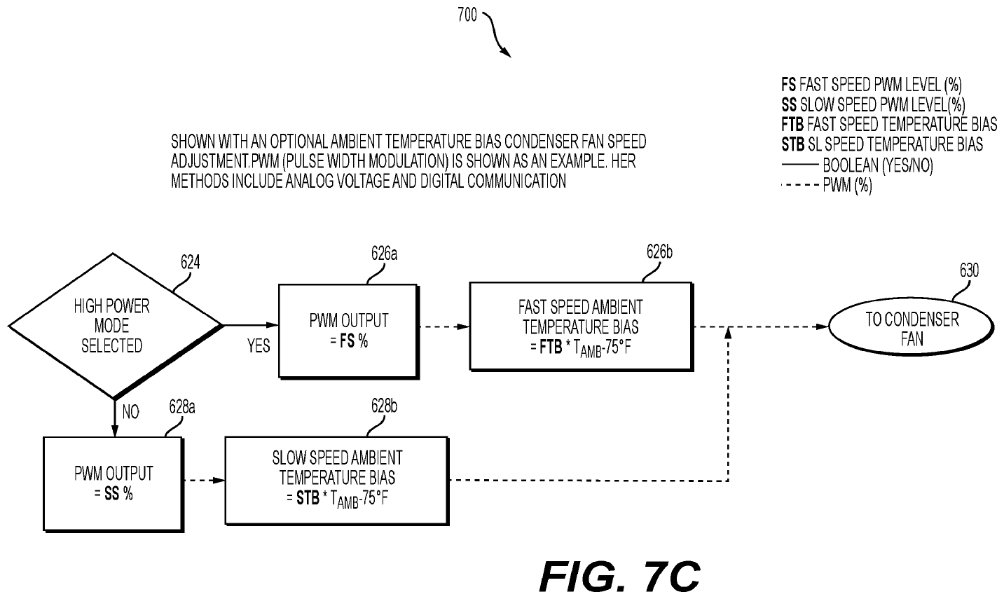

The control system 128 includes control logic 136 (see FIG. 7) that controls the operation of the components of the thermal regulation system 110. The control system 128 also includes one or more connector 130 (shown as “common connector” 130) and a power supply detection circuit or communication interface 132 (shown as “Power Supply Detection/Comm.” 132) that can connect to a set of power supplies 108 that is coupled to an AC source (e.g., wall outlet) or DC source (e.g., batteries). The power supply can include a DC-AC converter and/or AC/AC converter to provide power for the fluid circulation therapy device 102 and controller 104. Each power supply 108 includes a power supply connector 134 (shown as 134a, 134b, 134c) that connects to the common connector 130. Connector 134 includes an identification circuit that provides the detected connection 106 to the power supply detection circuit or communication interface 132. The power supply detection circuit 132 may include an analog to digital converter to detect resistance values of the connection. In other embodiments, the power supply detection circuit 132 may include a communication module that can serially communicate with a corresponding communication module in connector 134.

The detected connection 106 allows controller 104 to determine the connected power supply as a high-power mode power supply or a low-power mode power supply. That is, controller 104 can set its control logic 136 or parameters in control loop 138 for the controls of the fluid circulation therapy device 102 based on the detected connection 106. For example, controller 104 can adjust the parameter values, e.g., for maximum compressor speed or current limits, in its temperature control algorithm for the fluid circulation therapy device 102. In other embodiments, the detected connection 106 allows the controller 104 to set its control logic for other modes of operations by determining the connected power supply as a normal-noise mode power supply, low noise-mode power supply. For example, controller 104 can adjust parameter values for fan speed, compressor speed, etc., for normal or low-noise operation.

In some embodiments, the power supply 108 may include power pins and an additional Boolean “+” or “−” non-power connection pin in its cabling that terminates at the connector 134. An example would be the inclusion of a pin “5” in a 5-pin power connector that also has a “+” pin for a high-power supply and − for a low-power supply. Other appropriate mechanical electrical connection may be used.

In a further example, if more than two levels of power supply defined operating mode is desired, an analog or digital detection method could be used. For analog detection, different resistor values (ohms) between the power pin and either + or − can be employed. For digital detection, digital communication “handshake” circuits between the power supply and device can be employed.

The controller 104 may include a driver to provide a control output or power output to the compressor 114 or pump 112 of the fluid circulation therapy device 102. The driver can include a pulse width modulation (PWM) controller, an analog-digital converter (ADC), an isolation transformer, a filter, and/or additional circuitry.

In its most basic configuration, a controller 104 includes at least one processing unit and system memory. Depending on the exact configuration and type of computing device, system memory may be volatile (such as random-access memory (RAM)), non-volatile (such as read-only memory (ROM), flash memory, etc.), or some combination of the two. The processing unit may be a standard programmable processor that performs arithmetic and logic operations necessary for the operation of the computing device. As used herein, processing unit and processor refers to a physical hardware device that executes encoded instructions or logic for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

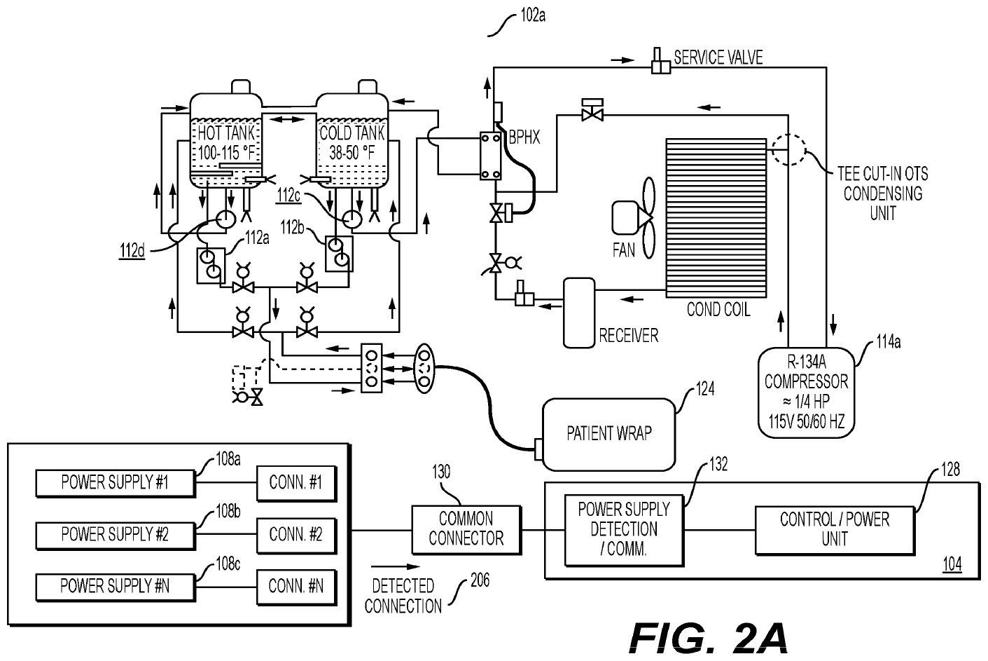

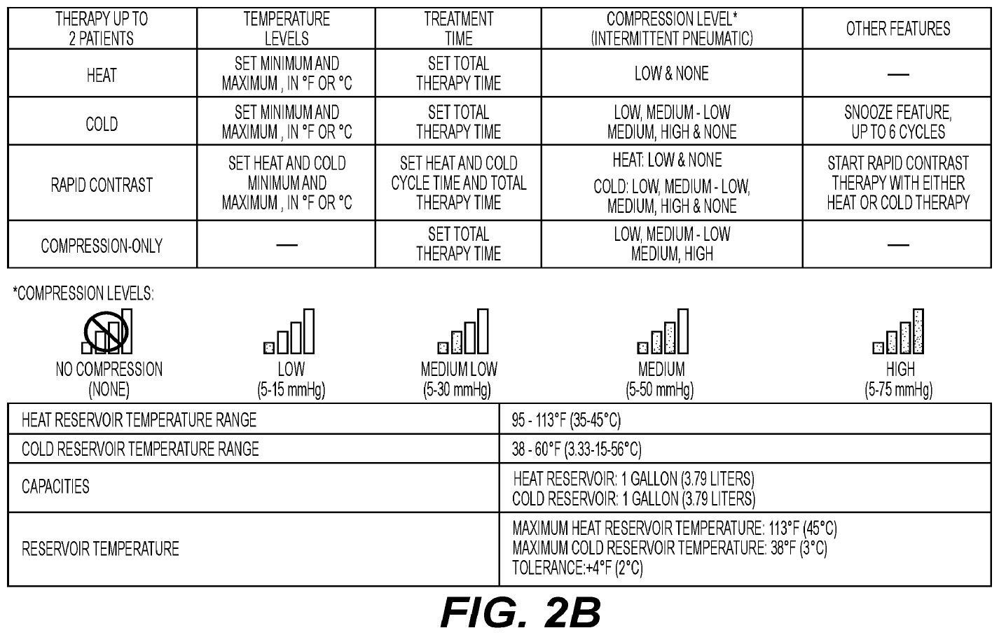

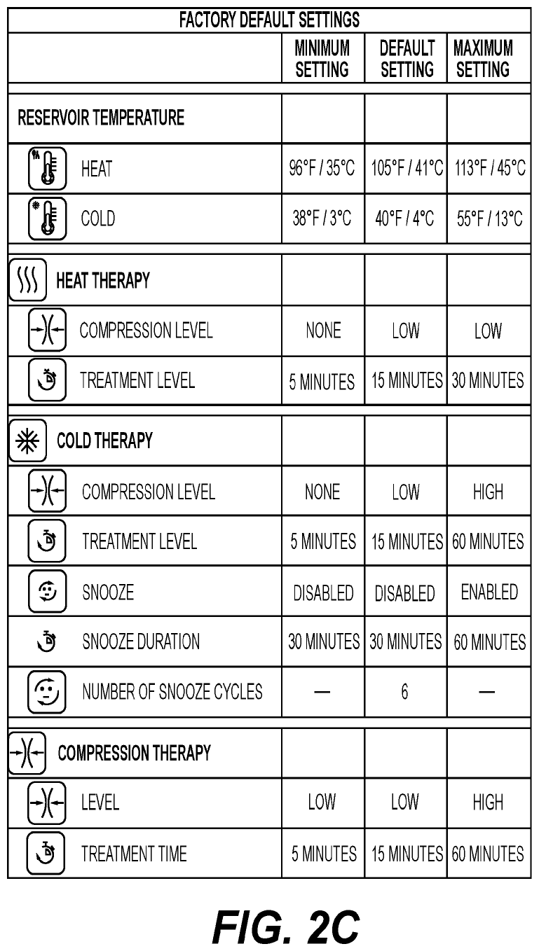

FIG. 2A shows an example embodiment of fluid circulation system 102 (shown as 102a) in accordance with an illustrative embodiment. In the example shown in FIG. 2A, the fluid circulation system 102a operates with a controller 104 that is configured to drive a compressor 114a. Based on detected connection 106 (shown as 206) from the power supply 108, via the power supply detection unit 132, controller 104 is configured to set different operating parameters or logic.

FIG. 2A is a schematic diagram of a fluid circulation therapy device 102 (shown as 102a) comprising an AC system and a variable speed DC compressor 114 (shown as 114a). The compressor 114a can be operated in lower power to allow the use of a heater in the cold tank. Device 102 includes pump 112 (shown as 112a, 112b, 112c, 112d) that can be used to pump fluid to the chiller, the heater, between the cold tank and the hot tank, and to the patient wrap 124. For example, a recirculation pump can be used to direct flow from the cold tank to the hot tank, and recirculation pump can be used to direct flow from the hot tank to the cold tank. The pumps, in combination with a system of valves, can be used to control the fluid flow in the system. Additional descriptions of the fluid circulation therapy device 102, among other embodiments, are shown in U.S. Publication No. 2020/0000628.

The base temperature control may be a proportional-feedback loop or a proportional-integrator feedback loop. It is that certain fluid circulation therapy device 102 can include both a hot and a cold circulation loop. In other devices, the fluid circulation therapy device 102 can include only a cold loop.

FIGS. 2B and 2C show example screens displayed by the touch screen interface of the fluid circulation therapy device 102a and parameters for control logic and operation of the controller 104. The system 102a can include a controller and/or processor and memory for storing instructions and programming to implement the user interfaces described herein as well as controlling the system as described herein. The various components, such as the pumps, the sensors, the compressors, the heat exchangers, the heaters, and the valves, can be controlled by the processor and/or send information to the processor.

### Example Therapeutic Wraps

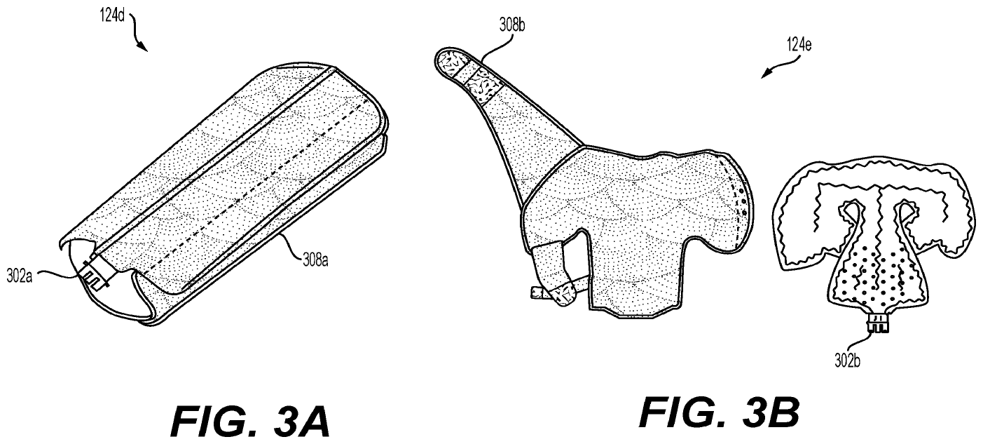

FIGS. 3A and 3B show additional examples of fluid wraps and compresses 124 (shown as 124d and 124e). The wrap and compress 124 are each configured with connectors 302 (shown as 302a and 302b) to connect over the circulation loop 122 to receive the therapeutic cooling or heating fluid from the fluid circulation therapy device 102. The fluid then circulates in internal fluid loops throughout the wrap or compress and then exits the wrap or compress through the connector 302 to return to the cooling and heating tanks of the fluid circulation therapy device 102 (see FIG. 2A). The wrap or compress 124 may also include an air-input port that can provide air to the wrap or compress to provide compression therapy. The fluid circulation and compression operation can be regulated or controlled by the controller 104. The fluid wrap or compress 124 may include straps such as a hook and loop fastener (shown as 308a and 308b) for attachment to the user.

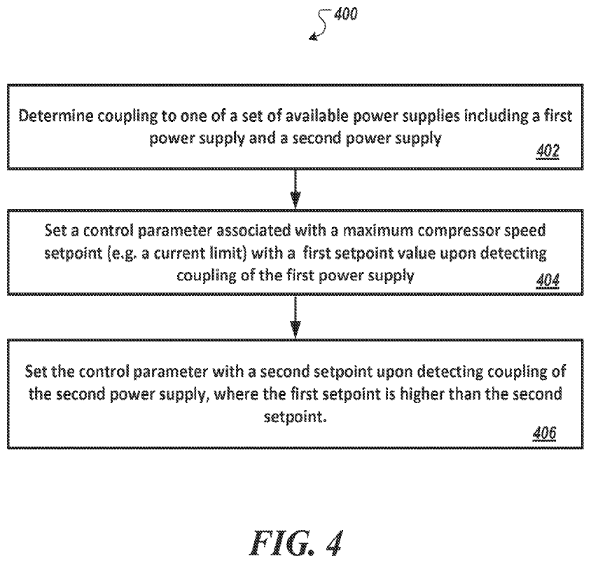

FIG. 4 shows a method 400 to operate a circulation therapy device (e.g., 102) with adaptive power supply-based control in accordance with an illustrative embodiment. Method 400 includes providing a compressor that is configured to connect in a fluid loop with a fluid circulation cooling therapy device, including a first fluid circulation therapy device (e.g., having a first cooling capacity) and a second fluid circulation therapy device (e.g., having a second cooling capacity). A controller is operatively coupled to the compressor, wherein the controller comprises a control output coupled to a driver that is configured to energize the compressor from a power source. Method 400 includes using a detection circuit configured to determine (402) the coupling to one of a set of available power supplies, including a first power supply and a second power supply, wherein the first power supply has a first power output, and the second power supply has a second power output, wherein the first voltage output is higher than the second voltage output (e.g., having higher voltage or current to provide higher power). Method 400 includes configuring the controller having instructions for a control loop of the compressor to determine the control output, wherein the control loop includes instructions to i) set (404) a control parameter associated with a maximum compressor speed setpoint (e.g., current limit) with a first setpoint value upon detecting coupling of the first power supply and ii) set (406) the control parameter with a second setpoint upon detecting coupling of the second power supply, and wherein the first setpoint is higher than the second setpoint.

Typically, operations 404 or 406 are performed once during the manufacturing or packaging of the circulation therapy device.

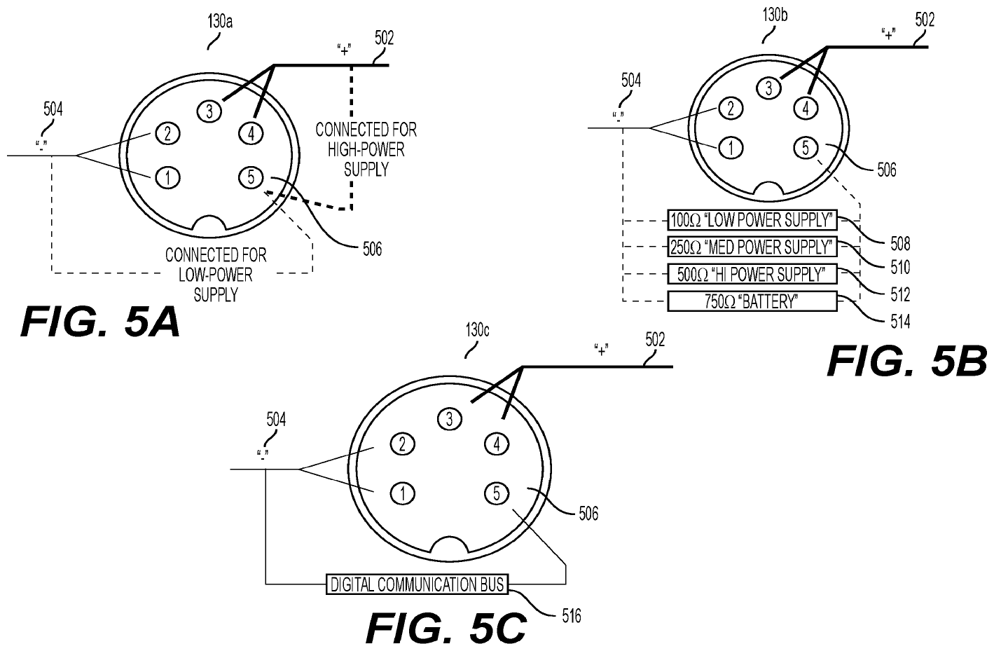

FIGS. 5A, 5B, 5C each shows example embodiments of the common connector 130 (shown as 130a, 130b, 130c) of the controller 104 to facilitate adaptive power supply-based control. In FIG. 5A, the connector 130a is configured, for example, as a DIN connector, having wires for (i) a first wiring configuration associated with the first power supply and (ii) a second wiring configuration associated with the second power supply. As noted above, if the connected power supply has a higher output voltage, it is deemed the high-power mode power supply, and if the power supply has wiring for a low-power mode, it is a low-power mode power supply.

In the example shown in FIG. 5A, pins 3 and 4 (502) of the connector (e.g., 130) are configured to connect to a high-power mode power supply, and pins 1 and 2 (504) of the connector (e.g., 130) are configured to connect to a low-power mode power supply. Pin 506 can serve as a neutral line for the high or low AC wire.

FIG. 5B shows an alternative embodiment of FIG. 5A in which the low power pins are configured with multiple resistance values. In this example, the power supply detection circuit 132 can first interrogate pins 1, 2, and 5 of the connector to determine one of an available set of resistance associated with the connection. In the example of FIG. 5B, the resistance of the connector 130 is set based on an internal resistor component: 100 Ω, 250 Ω, 500Ω, and 750Ω. The assessed resistance can be used to configure the controller 104 for operation with a low-power mode power supply 508, a medium-power mode power supply 510, a high-power mode power supply 512, or a battery source 514. The pins 3, 4 (502) and pins 1, 2 (504) can be used to carry the power to controller 104 to operate the fluid circulation therapy device 102.

FIG. 5C shows another alternative embodiment in which digital communication “handshake” between the power supply and controller 104 is employed to provide identification of the connected power supply. The digital communication 516 (shown as “digital communication bus” 516) may be based on serial communication protocol, such as CAN or SPI. In some embodiments, the digital communication may provide an encoded value for the connected power supply, e.g., “01” for a low-power mode power supply, “10” for a medium-power mode power supply, “11” a high-power mode power supply, and (“00”) for a battery source.

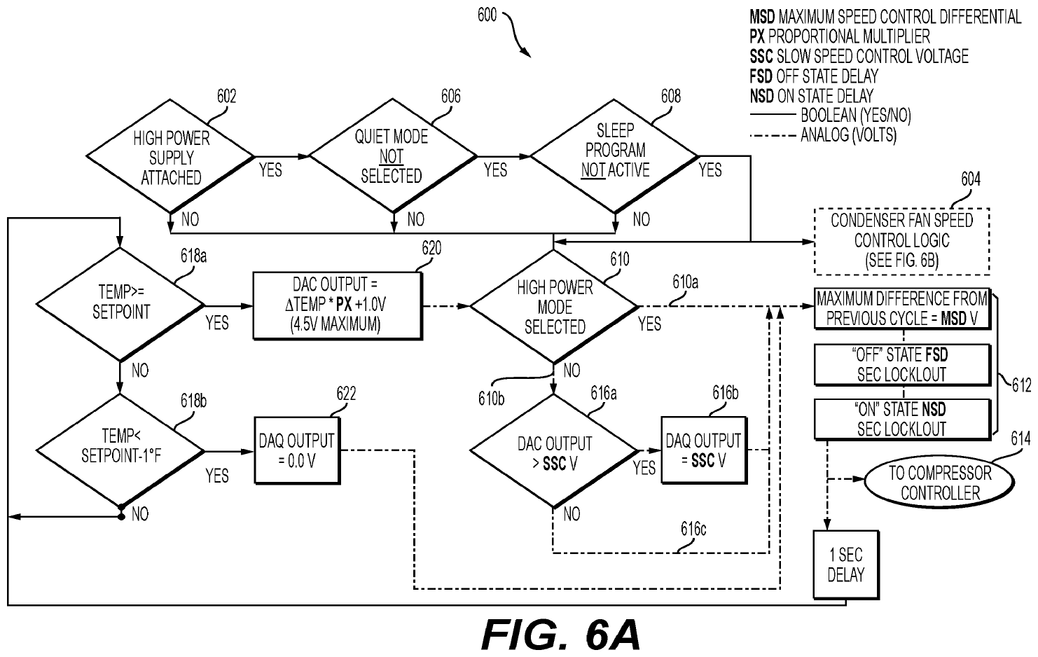

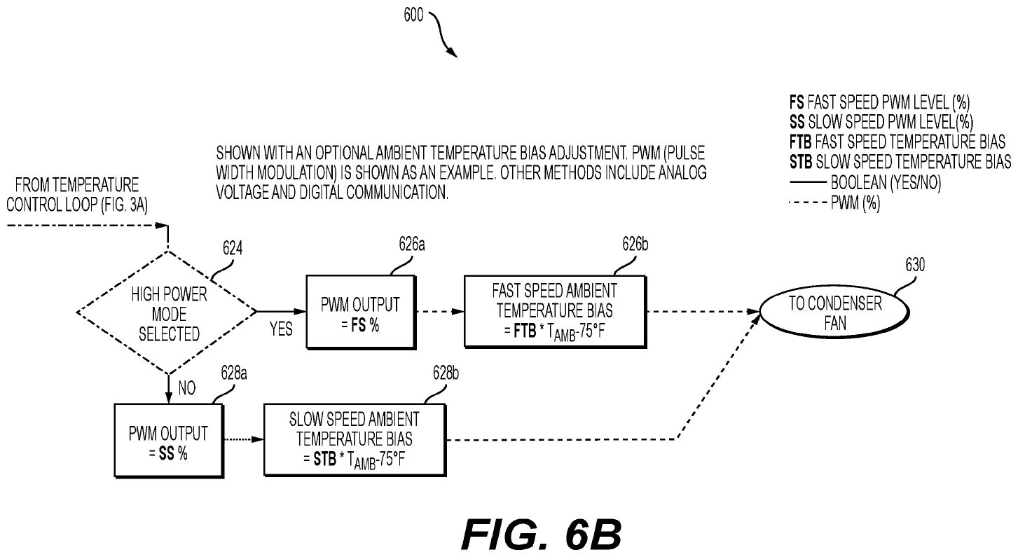

FIGS. 6A and 6B, collectively, show an example control loop and logic (138, 136) (shown as 600) for the controller 104 of FIG. 1 configured with adaptive power supply-based control. Control loop and logic 600 includes control nodes that can set condenser fan speeds and compressor speed, e.g., for the fluid circulation therapy device 102 of FIGS. 1, 2A.

In the example shown in FIG. 6, controller 104 first evaluates, using an input from the power supply detection circuit 132, at node 602, whether a high-power mode power supply is attached to the fluid circulation therapy device 102. Upon determination that the high-power mode power supply is connected, the control logic (604) for the condenser fan speed can be invoked (see FIG. 6B). Controller 104 can also evaluate whether the quiet mode (606) or sleep program mode (608) are selected.

Controller 104 then evaluates (610) if the high-power mode is selected (610a) from the user interface. If selected, controller 104 is configured to set the appropriate control parameters and setpoints (612) for high power operation and then execute the compressor control (614). If the high-power mode is not selected, controller 104 evaluates (616a) if the output for the compressor is currently set to greater than the slow speed control. If so, it sets (616b) the output as the slow speed control voltage value. If not (i.e., the output setpoint for the compressor is greater than the slow speed control voltage setpoint), the controller 104 sets (616c) at the appropriate control parameters and setpoints (612) for high power operation and then execute the compressor control (614).

Subsequent to compressor controls, the control loop and logic 600 executes the temperature control loops for the fluid circulation therapy device 102. The controller 104 evaluates (618a) if the sensed temperature of the circulation fluid is greater than the setpoint. If so (i.e., the temperature is higher than the setpoint), the control loop and logic 600 sets 620 the output to the compressor to increase the cooling using a proportional feedback control where the difference in temperature is multiplied by a proportional multiplier plus a constant subject to a maximum constraint. If the sensed temperature of the circulation fluid is less than the setpoint (618b), controller 104 is configured to set 622 to compressor output to zero. Table 1 provides a description of the parameters shown in FIGS. 6A and 6B.

TABLE 1

| | Parameter | |
|---|---|---|
| Parameter | Name | Parameter Description |
| PX | Proportional | This determines the slope of the setpoint |
| | Multiplier | deviation vs. power curve. |
| SSC | Slow Speed | This is a control speed voltage selected to |
| | Control | minimize noise and reduce current to allow the |
| | Voltage | use of a smaller power supply |
| MSD | Maximum | This limits the speed control voltage change |
| | Speed | from one cycle to the next when DAC output is |
| | Control | greater than or equal to 1.0 v. The purpose is to |
| | Differential | prevent excessive cycling of the control |
| | | voltage. |
| FSD | Off State | The “Off” State lockout is a delay from the |
| | Delay | time the compressor is turned off (DAC output |
| | | of zero) until it can be restarted (DAC output |
| | | greater than or equal to 1.0 v). |
| NSD | On State | The “On” State lockout is a delay from the |
| | Delay | time the compressor is turned on (DAC output |
| | | greater than or equal to 1.0 v) until it can be |
| | | turned off (DAC output of zero) |
| FS | Fast Speed | This value is the base high-power fan speed. It |
| | PWM Level | will be further adjusted based on ambient |
| | (%) | temperature. The intent is to reduce the fan |
| | | speed at lower ambient temperatures to |
| | | minimize noise and increase fan speed at |
| | | elevated ambient temperatures to maintain |
| | | a safe compressor discharge temperature. |
| SS | Slow Speed | This value is the base low-power fan speed. It |
| | PWM Level | will be further adjusted based on ambient |
| | (%) | temperature. |
| FTB | Fast Speed | This constant defines the slope of the PWM |
| | Temperature | ambient temperature bias curve. |
| | Bias (%/° F.) | |
| STB | Slow Speed | This constant defines the slope of the PWM |
| | Temperature | ambient temperature bias curve. |
| | Bias (%/° F.) | |

As shown in the example of FIG. 6A, the DAC output (e.g., set in 610, 620, 622) is subject to a maximum speed control differential constraint.

FIG. 6B shows the control loop and logic for variable condenser fan speed in a cold therapy device. The variable condenser speed operation may be used to increase noise reduction allowing the fluid circulation therapy device 102 to operate in quiet mode (606) or sleep mode (608). In the example shown in FIG. 6B, controller 104 evaluates (624) if a high-power mode operation is selected. If “yes” (i.e., high-power mode operation is selected), the PWM output to the condenser is set (626a) to fast speed PWM level (in percentage), and the fast speed ambient temperature bias is set (626b) to a fast speed bias value. If “no” (i.e., high-power mode not selected), the PWM output to the condenser is set (628a) to a slow speed PWM level (in percentage), and the fast speed ambient temperature bias is set (626b) to a slow speed bias value. The PWM output is then provided (630) to the output of the condenser fan.

Indeed, controller 104, based on the logic 600 of FIGS. 6A-6B, is configured to determine the control output to set a control parameter associated with a maximum compressor speed setpoint (e.g., current limit) based on a detected power supply.

FIGS. 7A, 7B, and 7C, collectively, show another example control loop and logic (138, 136) (shown as 700) for the controller 104 of FIG. 1 configured with adaptive power supply-based control. Control loop and logic 700 also include control nodes that can set condenser fan speeds and compressor speed, e.g., for the fluid circulation therapy device 102 of FIGS. 1, 2A.

In the example shown in FIG. 7A, controller 104 first evaluates using an input from the power supply detection circuit 132, at node 702, whether a high-power mode power supply is attached to the fluid circulation therapy device 102. Upon determination that the high-power mode power supply is connected, the controller 104 sets (704) the parameters associated with condenser operation to the high-power base speed value (“HPS”). Otherwise, the controller 104 sets (706) the parameters associated with compressor operation to the low-power base speed value (“LPS”). Controller 104 can also evaluate (708) whether quiet or sleep mode is active. Upon determination that the sleep mode or quiet mode is selected, the controller 104 sets (710) the parameters associated with the fan operation to the quiet/sleep speed value (“QSS”).

The controller 104 can then initialize (712) the parameter value for the compressor operation to the maximum compressor speed, which can be adjusted based on sensed ambient temperature (block 714) or based on sensed current limit (block 716) prior to the compressor output values being provided (718) to the control loop.

FIG. 7B shows a similar control logic for the fluid circulation therapy device 102, as shown in FIG. 6A. The control loop and logic 700 execute the temperature control loops for the fluid circulation therapy device 102. The controller 104 evaluates (618a) if the sensed temperature of the circulation fluid is greater than the setpoint. If so (i.e., the temperature is higher than the setpoint), the control loop and logic 600 sets 620 the output to the compressor to increase the cooling using a proportional feedback control where the difference in temperature is multiplied by a proportional multiplier plus a constant subject to a maximum constraint. If the sensed temperature of the circulation fluid is less than the setpoint (618b) by an offset, the controller 104 is configured to set (622a, 622b) to compressor output to zero or to a minimum value output (shown as “1.0V”). Notably, the DAC output for the compressor is constrained by the maximum compressor speed values set in the logic shown in FIG. 7A. FIG. 7C shows a similar control logic for the fluid circulation therapy device 102, as shown in FIG. 6B.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present.

In contrast, when a feature or element is referred to as being “directly connected,” “directly attached,” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed of “adjacent” another feature may have portions that overlap or underlie the adjacent feature.

The terminology used herein is for the purpose of describing particular embodiments only and is not intended to be limiting of the invention. For example, as used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/.”

Spatially relative terms, such as “under,” “below,” “lower,” “over,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations), and the spatially relative descriptors used herein interpreted accordingly. Similarly, the terms “upwardly,” “downwardly,” “vertical,” “horizontal,” and the like are used herein for the purpose of explanation only unless specifically indicated otherwise.

Although the terms “first” and “second” may be used herein to describe various features/elements (including steps), these features/elements should not be limited by these terms unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention. Throughout this specification and the claims which follow, unless the context requires otherwise, the word “comprise,” and variations such as “comprises” and “comprising” means various components can be co-jointly employed in the methods and articles (e.g., compositions

and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed, the “less than or equal to X” as well as “greater than or equal to X” (e.g., where X is a numerical value) is also disclosed. It is also understood that throughout the application, data is provided in a number of different formats and that this data represents endpoints and starting points and ranges for any combination of the data points.

For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments, one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The invention claimed is:

1. A fluid circulation therapy system comprising:
a compressor configured to connect in a fluid loop with a fluid circulation cooling therapy device, including a first fluid circulation therapy device and a second fluid circulation therapy device;
controller operatively coupled to the compressor, wherein the controller comprises a control output coupled to a driver that is configured to energize the compressor from a power source; and
a detection circuit configured to determine coupling to one of a set of available power supplies as a connected power supply, including a first power supply and a second power supply, wherein the first power supply has a first power output, and the second power supply has a second power output, wherein the first power output is higher than the second power output;
the controller having instructions for a control loop of the compressor to determine the control output, wherein the control loop includes instructions to (i) set a control parameter associated with a maximum compressor speed setpoint with a first setpoint value upon detecting coupling of the first power supply and (ii) set the control parameter with a second setpoint upon detecting coupling of the second power supply, and wherein the first setpoint is higher than the second setpoint.

2. The fluid circulation therapy system of claim 1, wherein the detection circuit is configured to determine coupling to the one of the set of available power supplies via an analog or digital output or a sensed electrical characteristic provided by the connected power supply at a power supply cable that connects the connected power supply to the detection circuit.

3. The fluid circulation therapy system of claim 1, wherein the power supply cable comprises a connector, the connector having wires for (i) a first wiring configuration associated with the first power supply and (ii) a second wiring configuration associated with the second power supply.

4. The fluid circulation therapy system of claim 1, wherein the power supply cable comprises a connector, the connector having wires for a digital communication bus to communicate with the first power supply or the second power supply.

5. The fluid circulation therapy system of claim 1, wherein the power supply cable comprises a connector, the connector having wires for (i) a first wiring configuration associated with the first power supply and (ii) a second wiring configuration associated with one of a set of available second power supplies or a battery.

6. The fluid circulation therapy system of claim 1, further comprising:
a variable condenser fan speed,
wherein the controller is coupled to the variable condenser fan speed, wherein the controller comprises a second control output coupled to the driver that is configured to energize the variable condenser fan speed from the power source; and
wherein the controller has instructions for a control loop of the variable condenser fan speed to determine the second control output, wherein a second control loop for the second control output includes instructions to i) set a control parameter associated with a maximum variable condenser fan speed setpoint with a third setpoint value upon detecting coupling of the first power supply and ii) set the control parameter with a fourth setpoint upon detecting coupling of the second power supply, and wherein the third setpoint is higher than the fourth setpoint.

7. The fluid circulation therapy system of claim 1, wherein the control loop includes instructions to set the control parameter associated with a maximum compressor speed setpoint with a third setpoint for operation of the compressor that minimize acoustic noise.

8. The fluid circulation therapy system of claim 1, wherein the control loop includes instructions to select a setting to minimize noise and/or reduce current.

9. The fluid circulation therapy system of claim 1, wherein the control loop includes instructions to limit a speed control voltage change from one cycle to a next cycle.

10. The fluid circulation therapy system of claim 1, wherein the control loop includes instructions to:
provide a delay from a time the compressor is turned off to when it can be restarted; and
provide a delay from a time the compressor is turned on to when it can be turned off.

11. The fluid circulation therapy system of claim 1, wherein the first fluid circulation cooling therapy device is configured with a first cooling capacity and is configured to be attachable to a first area of a body, and wherein the second fluid circulation cooling therapy device is configured with a second cooling capacity and is configured to be attachable to a second area of the body.

12. The fluid circulation therapy system of claim 1, wherein the first fluid circulation therapy device is configured to be placed at a first body region of a user, and the second fluid circulation therapy device is configured to be placed at a second body region of the user, wherein the first body region is different from the second body region.

13. The fluid circulation therapy system of claim 12, wherein the first body region or the second body region includes arm, shoulder, neck region, back, wrist, ankle, knee, leg, elbow, or foot.

14. A system comprising:
a controller configured to operatively couple to a compressor, wherein the compressor is configured to connect in a fluid loop with a fluid circulation cooling therapy device, including a first fluid circulation therapy device and a second fluid circulation cooling therapy device, and wherein the controller comprises a control output coupled to a driver that is configured to energize the compressor from a power source; and
a detection circuit configured to determine coupling to one of a set of available power supplies, including a first power supply and a second power supply, wherein the first power supply has a first power output, and the second power supply has a second power output, wherein the first power output is higher than the second power output;
the controller having instructions for a control loop of the compressor to determine the control output, wherein the control loop includes instructions to i) set a control parameter associated with a maximum compressor speed setpoint with a first setpoint value upon detecting coupling of the first power supply and ii) set the control parameter with a second setpoint upon detecting coupling of the second power supply, and wherein the first setpoint is higher than the second setpoint.

15. The system of claim 14, wherein the detection circuit is configured to determine coupling to the one of the set of available power supplies via an analog or digital output or a sensed electrical characteristic provided by the connected power supply at a power supply cable that connects the connected power supply to the detection circuit.

16. The system of claim 14, wherein the power supply cable comprises a connector, the connector having wires for at least one of:
i) a first wiring configuration associated with the first power supply and ii) a second wiring configuration associated with the second power supply;
a digital communication bus to communicate with the first power supply and the second power supply; or
iii) a first wiring configuration associated with the first power supply and iv) a second wiring configuration associated with one of a set of available second power supplies or a battery.

17. The system of claim 14, wherein the control loop includes instructions to set the control parameter associated with a maximum compressor speed setpoint with a third setpoint for operation of the compressor that minimize acoustic noise.

18. The system of claim 14, wherein the first fluid circulation therapy device is configured to be placed at a first body region of a user, and the second fluid circulation therapy device is configured to be placed at a second body region of the user.

19. A method comprising:
detecting, via a circuit, coupling of a power supply cable between a controller and one of a set of available power supplies, including a first power supply and a second power supply, wherein the first power supply has a first power output, and the second power supply has a second power output, wherein the first voltage output is higher than the second voltage output;
setting, by a processor of the controller, a control parameter associated with a maximum compressor speed setpoint with a first setpoint value upon detecting coupling of the first power supply and ii) set the control parameter with a second setpoint upon detecting coupling of the second power supply, and wherein the first setpoint is higher than the second setpoint; and
outputting, by the controller, a control output for a compressor of a fluid circulation cooling therapy system, wherein the control output is limited by the control parameter associated with a maximum compressor speed setpoint.

20. The method of claim 19, wherein the power supply cable comprises a connector, the connector having wires for at least one of:
i) a first wiring configuration associated with the first power supply and ii) a second wiring configuration associated with the second power supply;
a digital communication bus to communicate with the first power supply and the second power supply; or
iii) a first wiring configuration associated with the first power supply and iv) a second wiring configuration associated with one of a set of available second power supplies or a battery.

21. The method of operating the system of claim 1.

22. A non-transitory computer-readable medium having instructions stored thereon, wherein execution of the instructions by a processor causes the processor to operate the system of claim 1.
